(19) United States
(12) Patent Application Publication
(10) Pub. No.: US 2018/0236257 A1
(43) Pub. Date: Aug. 23, 2018

(54) CELL STIMULATION METHOD AND CELL STIMULATION DEVICE

(71) Applicant: HAMAMATSU PHOTONICS K.K., Hamamatsu-shi (JP)

(72) Inventors: Gen TAKEBE, Hamamatsu-shi (JP); Yoshiyuki SHIMIZU, Hamamatsu-shi (JP); Toyohiko YAMAUCHI, Hamamatsu-shi (JP); Tatsuo DOUGAKIUCHI, Hamamatsu-shi (JP)

(73) Assignee: HAMAMATSU PHOTONICS K.K., Hamamatsu-shi (JP)

(21) Appl. No.: 15/896,267

(22) Filed: Feb. 14, 2018

(30) Foreign Application Priority Data
Feb. 17, 2017 (JP) 2017-028025

(51) Int. Cl. A61N 5/06; C12M 1/42; C12N 13/00

(52) U.S. Cl. CPC A61N 5/062; C12M 35/02; C12N 2529/10; A61N 2005/0659; C12N 13/00

(57) ABSTRACT

A cell stimulation method includes continuously emitting mid-infrared light to a living cell and thus changing an ion concentration of the cell or changing ion concentrations of the cell and other cells disposed around the cell.

### TECHNICAL FIELD

The present disclosure relates to a cell stimulation method and a cell stimulation device.

### BACKGROUND

For example, in Non-Patent Literature 1 and Non-Patent Literature 2, methods of changing a calcium ion (Ca²⁺) concentration of a cell using near infrared light are described. In the method described in Non-Patent Literature 1, metal particles are disposed in the vicinity of Hela cells in a culture plate, near infrared light with a wavelength of 1064 nm is emitted to the metal particles, heat is thus generated from the metal particles, and the Ca³⁺ concentration of the Hela cells is changed due to the heat of the metal particles. In the method described in Non-Patent Literature 2, near infrared pulse light is directly emitted to myocardial cells in a culture plate, and thus the Ca²⁺ concentration of the myocardial cells is changed. In this method, near infrared pulse light with a wavelength of 1862 nm, a pulse energy of 9.1 J/cm² to 11.6 J/cm², and a pulse width of 3 ms to 4 ms is used.

[Non-Patent Literature 1] Vadim Tseeb, Madoka Suzuki, Kotaro Oyama, Kaoru Iwai, Shin'ichi Ishiwata, “Highly thermosensitive Ca²⁺ dynamics in a HeLa cell through IP3 receptors,” HFSP (Human Frontier Science Program) Journal, 21 Oct. 2008, pp 117-123.

[Non-Patent Literature 2] Gregory M Dittami, Suhrud M Rajguru, Richard A Lasher, Robert Whitchcock, Richard D Rabbitt, “Intracellular calcium transients evoked by pulsed infrared radiation in neonatal cardiomyocytes,” The Journal of Physiology, 15 Mar. 2011, pp 1295-1306.

### SUMMARY

Biological cells include organic molecules (biomolecules) such as nucleic acids, proteins, lipids, and sugars. Functional groups of such biomolecules and bonds between the biomolecules have vibrations specific to the biomolecules. When infrared light is emitted to such biomolecules, the biomolecules absorb infrared light photon energy. A magnitude of infrared light photon energy absorbed by the biomolecules corresponds to a magnitude of energy necessary to change a vibration state of the biomolecules. Therefore, when infrared light is emitted to biomolecules, it is possible to change a vibration state of the biomolecules. Such a change in the vibration state of the biomolecules is thought to cause a change in an ion concentration of the biomolecules. For example, a method in which the Ca²⁺ concentration of cells is changed using near infrared light has been conceived (for example, refer to Non-Patent Literature 1 and Non-Patent Literature 2).

However, since not much light with a wavelength in a near infrared range is absorbed by biomolecules, the methods described in Non-Patent Literature 1 and Non-Patent Literature 2 have the following problems.

That is, in the method described in Non-Patent Literature 1, near infrared light is not directly emitted to cells, but near infrared light is emitted to metal particles in the vicinity of the cells. Accordingly, in this method, it is difficult to efficiently change an ion concentration of cells compared to when near infrared light is directly emitted to cells. In addition, in this method, since it is necessary to provide metal particles in the vicinity of cells, there is a possibility of this method not being able to be applied to, for example, cells in a living body.

In the method described in Non-Patent Literature 2, in order to change an ion concentration of cells, the pulse energy of near infrared pulse light needs to have a certain magnitude. That is, when a magnitude of the pulse energy of the near infrared pulse light is reduced, there is a possibility of an ion concentration of cells not being changed. Therefore, it is difficult to efficiently change an ion concentration of cells using such near infrared pulse light. In addition, in this method, when emission of near infrared pulse light to cells continues, there is a risk of the cells being damaged or killed. Therefore, in such a case, there is a possibility of an ion concentration of living cells not being changed.

The present disclosure has been made in order to address such problems, and an object of the present disclosure is to provide a cell stimulation method and a cell stimulation device through which it is possible to efficiently change an ion concentration of living cells.

In the cell stimulation method according to an embodiment of the present disclosure, when mid-infrared light is continuously emitted to living cells, an ion concentration of cells is changed or ion concentrations of cells and other cells disposed around the cells are changed.

The cell stimulation device according to an embodiment of the present disclosure includes a light emission unit configured to output mid-infrared light. When mid-infrared light is continuously emitted to living cells, an ion concentration of cells is changed or ion concentrations of cells and other cells disposed around the cells are changed.

As described above, methods in which an ion concentration in cells is changed using near infrared light within infrared light have been proposed. However, since not much light with a wavelength in a near infrared range is absorbed by biomolecules, it is difficult to efficiently change an ion concentration of living cells using near infrared light. On the other hand, the inventors focused on the fact that a wavelength range of mid-infrared light within infrared light corresponds to a fingerprint range of biomolecules (that is, a wavelength range in which the intrinsic absorption peaks of biomolecules appear) and is a wavelength range in which absorption into biomolecules in cells is greatest, and found that, when mid-infrared light is directly emitted to cells, it is possible to efficiently change an ion concentration of living cells. Specifically, since many intrinsic absorption peaks of biomolecules in cells appear in the wavelength range of mid-infrared light, when mid-infrared light having a wavelength corresponding to an absorption band of a certain specific biomolecule is emitted to cells, it is possible to change an ion concentration of an arbitrary biomolecule in cells. On the other hand, in contrast to near infrared light, since absorption into biomolecules in cells is greatest in mid-infrared light, even if an emission intensity of mid-infrared light is reduced to become lower than an emission intensity (an emission intensity necessary for changing an ion concentration of cells) of near infrared light, it is possible to change an ion concentration of cells. In addition, since an emission intensity of mid-infrared light can be reduced in this manner, even if mid-infrared light is continuously emitted to cells, it is possible to change an ion concentration of cells while avoiding damage to cells or cell death. Therefore, it is possible to sustainably change an ion concentration of cells.

Mid-infrared light may be emitted to a part of a cell. When mid-infrared light is locally emitted to a part of the cell, a change in ion concentration that is different from a change in ion concentration in a part other than that part of the cell can be caused in that part of the cell.

That is, it is possible to locally change an ion concentration of the cell.

A wavelength of mid-infrared light may be 4 μm or more and 10 μm or less. Many intrinsic absorption peaks of biomolecules in cells appear particularly in this wavelength range. Accordingly, it is possible to suitably obtain the above-described effects of the present disclosure.

According to the embodiment of the present disclosure, it is possible to efficiently change an ion concentration of living cells.

### BRIEF DESCRIPTION OF THE DRAWINGS

FIG. 1 is a schematic configuration diagram of a cell stimulation device according to an embodiment.

FIG. 2 is a flowchart showing a cell stimulation method according to an embodiment.

FIG. 3A, FIG. 3B, FIG. 3C, and FIG. 3D are images showing a fluorescence intensity in a first example.

FIG. 4A is a graph showing change in a fluorescence intensity over time in the first example. FIG. 4B is a graph showing a part in FIG. 4A enlarged.

FIG. 5 is an image showing a fluorescence intensity in a second example.

FIG. 6 is a graph showing change in a fluorescence intensity over time in the second example.

FIG. 7 is an image showing a fluorescence intensity in a third example.

FIG. 8 is a graph showing change in a fluorescence intensity over time in the third example.

FIG. 9 is an image showing a fluorescence intensity in a fourth example.

FIG. 10 is a graph showing change in a fluorescence intensity over time in the fourth example.

### DETAILED DESCRIPTION

A cell stimulation device and a cell stimulation method according to embodiments of the present disclosure will be described below in detail with reference to the appended drawings. Components in descriptions of the drawings which are the same are denoted with the same reference numerals, and redundant descriptions thereof will be omitted.

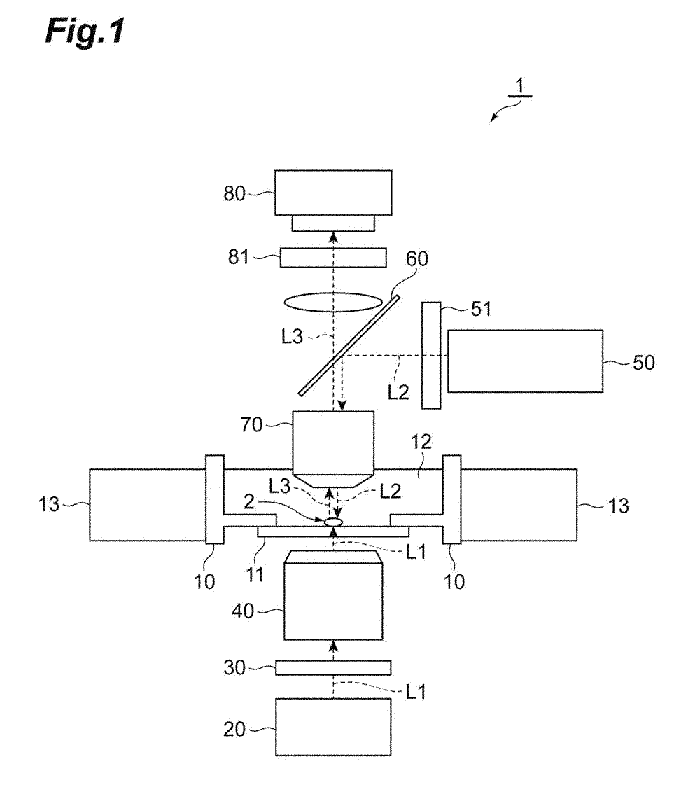

FIG. 1 is a schematic configuration diagram of a cell stimulation device 1 according to an embodiment of the present invention. The cell stimulation device 1 is a device configured to emit mid-infrared light L1 to living cells 2 so that an ion concentration of the cells 2 is changed, and changes in the ion concentration are successively observed. As shown in FIG. 1, the cell stimulation device 1 includes a culture plate 10, an infrared light source (light emission unit) 20, a shutter 30, an objective lens 40, an excitation light source 50, a dichroic mirror 60, an objective lens 70, and an imaging device 80.

The culture plate 10 includes a silicon wafer 11. An opening is provided on a bottom surface of the culture plate 10, and the silicon wafer 11 is attached to the opening so that the opening is closed. The cells 2 are disposed on the silicon wafer 11. On the culture plate 10, a culture solution 12 is accepted together with the cells 2 on the silicon wafer 11. The cells 2 are, for example, Hela cells (cells derived from cervical cancer), CHO cells (Chinese hamster ovary cells), or Neuro-2a (mouse ganglioneuroblastoma). A dyeing treatment using a fluorescent reagent may be performed on the cells 2. When the dyeing treatment is performed on the cells 2, the fluorescent reagent is incorporated into the cells 2. The fluorescent reagent quantitatively reacts with specific ions of the cells 2 and emits fluorescence L3. An intensity of the fluorescence L3 is proportional to an ion concentration of the cells 2. Examples of ions of the cells 2 include calcium ions (Ca³⁺ ), sodium ions (Na³⁺ ), potassium ions (K⁺), chlorine ions (Cl⁻), magnesium ions (Mg³⁺ ), and zinc ions (Zn²⁺). Here, a specific method of the dyeing treatment of the cells 2 will be described in a first example and a fourth example to be described below. A heater 13 is provided around the culture plate 10. The heater 13 is attached to surround the outer circumferential surface of the culture plate 10. The heater 13 is provided to keep the cells 2 in the culture plate 10 at a predetermined temperature (for example, 36° C. which is a body temperature).

The infrared light source 20 is positioned below the culture plate 10. The infrared light source 20 outputs mid-infrared light L1 toward the cells 2 in the culture plate 10. The mid-infrared light L1 is continuous light. Continuous light here includes not only light that is continuously output without a time interval but also pulse light that is repeatedly output with a time interval of 1 kHz or more. This is because, since a time width of an action potential (spike) of, for example, nerve cells, is about 1 millisecond, it can be assumed that there will be no difference in change of state of an ion concentration of the cells 2 between a case in which pulse light that is output at time intervals shorter than this time width is emitted to the cells 2 and a case in which light that is continuously output without a time interval is emitted to the cells 2. The optical axis of the mid-infrared light L1 extends, for example, in a direction perpendicular to a bottom surface of the culture plate 10. For example, the wavelength of the mid-infrared light L1 is appropriately in a range of 4 μm to 10 μm. This is because this wavelength range corresponds to a range (fingerprint range) in which many intrinsic absorption peaks of biomolecules in the cells 2 appear.

The objective lens 40 is positioned between the infrared light source 20 and the culture plate 10, and is disposed to face a back surface (specifically, a surface opposite to a surface on which the cells 2 are disposed) of the silicon wafer 11 of the culture plate 10. The objective lens 40 is optically coupled to the infrared light source 20. The objective lens 40 condenses the mid-infrared light L1 emitted from the infrared light source 20 on the cells 2 on the silicon wafer 11. The mid-infrared light L1 emitted from the objective lens 40 is emitted to the back surface of the silicon wafer 11, passes through the silicon wafer 11, and is emitted to the cells 2 on the silicon wafer 11.

The shutter 30 is positioned between the infrared light source 20 and the objective lens 40, and provided along the optical axis of the mid-infrared light L1. The shutter 30 can be opened or closed. An emission period during which the mid-infrared light L1 is emitted to the cells 2 and a non-emission period during which the mid-infrared light L1 is not emitted to the cells 2 are adjusted according to opening and closing timings of the shutter 30. In a period in which the shutter 30 is open (hereinafter this period will be referred to as an “emission period”), the mid-infrared light L1 output from the infrared light source 20 passes through the shutter 30, and is then continuously emitted to the cells 2 in the culture plate 10 through the objective lens 40. On the other hand, in a period in which the shutter 30 is closed (hereinafter this period will be referred to as a “non-emission period”), the mid-infrared light L1 is blocked by the shutter 30 so that it is not emitted to the cells 2 in the culture plate 10.

The excitation light source 50, the dichroic mirror 60, the objective lens 70, and the imaging device 80 are positioned above the culture plate 10. The dichroic mirror 60, the objective lens 70, and the imaging device 80 are disposed in an optical axis direction of the mid-infrared light L1. The excitation light source 50 is disposed in a direction crossing an optical axis direction of the mid-infrared light L1. The excitation light source 50 is provided to emit excitation light L2 to the cells 2 in the culture plate 10. The excitation light source 50 outputs visible light toward the dichroic mirror 60. Visible light includes an excitation wavelength at which the fluorescent reagent in the cells 2 can be excited. An excitation filter 51 is provided along the optical axis of the visible light. The excitation filter 51 selectively transmits the excitation light L2 with a specific wavelength within visible light received from the excitation light source 50 and blocks light with other wavelengths. When the excitation light L2 is emitted to the cells 2, the fluorescent reagent in the cells 2 is excited, the fluorescence L3 with a predetermined wavelength is emitted from the cells 2.

The dichroic mirror 60 is attached between the culture plate 10 and the imaging device 80 at a position at which it crosses the optical axis of the mid-infrared light L1 and the optical axis of the excitation light L2. The dichroic mirror 60 is provided so that a surface thereof is oblique to the optical axis of the excitation light L2 and the optical axis of the mid-infrared light L1. The dichroic mirror 60 has wavelength band characteristics in which light with a wavelength shorter than a specific wavelength is reflected, but light with a wavelength equal to or greater than the specific wavelength is transmitted. The dichroic mirror 60 reflects the excitation light L2 received from the excitation filter 51 toward the cells 2 in the culture plate 10, and transmits the fluorescence L3 emitted from the cells 2.

The objective lens 70 is disposed between the dichroic mirror 60 and the silicon wafer 11 of the culture plate 10. The objective lens 70 is optically coupled to the excitation light source 50 through the dichroic mirror 60. The objective lens 70 condenses the excitation light L2 received from the dichroic mirror 60. In addition, the objective lens 70 collimates the fluorescence L3 emitted from the cells 2 and emits it toward the imaging device 80. A fluorescent filter 81 is provided between the objective lens 70 and the imaging device 80. The fluorescent filter 81 selectively transmits the fluorescence L3 emitted from the objective lens 70 and blocks light with other wavelengths. The imaging device 80 receives the fluorescence L3 transmitted by the fluorescent filter 81 and acquires an image of the fluorescence L3.

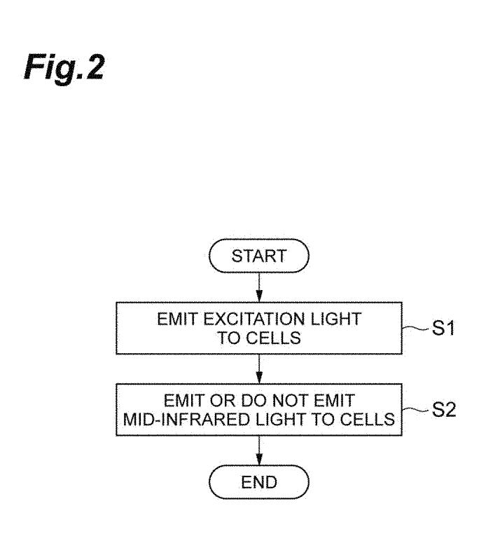

Next, operations of the cell stimulation device 1 will be described. In addition, a cell stimulation method according to the present embodiment will be described. FIG. 2 is a flowchart showing a cell stimulation method. First, the excitation light L2 output from the excitation light source 50 passes through the excitation filter 51, is reflected by the dichroic mirror 60, and is then emitted to the cells 2 in the culture plate 10 through the objective lens 70 (Step S1). When the excitation light L2 is emitted to the cells 2, the fluorescence L3 with an intensity corresponding to the ion concentration according to the fluorescent reagent is emitted from the cells 2. The fluorescence L3 emitted from the cells 2 is collimated by the objective lens 70 and then passes through the dichroic minor 60 and the fluorescent filter 81 and reaches the imaging device 80. Next, the infrared light source 20 outputs the mid-infrared light L1 toward the cells 2 in the culture plate 10. In the emission period, the mid-infrared light L1 is continuously emitted to the cells 2 in the culture plate 10 through the objective lens 40. In the non-emission period, the mid-infrared light L1 is blocked by the shutter 30 and is not emitted to the cells 2 in the culture plate 10 (Step S2). Changes in state of the intensity of the fluorescence L3 in the emission period or the non-emission period are successively observed by the imaging device 80.

The present disclosure will be described below in detail with reference to the first example to the fourth example. In the following first example to the fourth example, a water immersion objective lens having a magnification of 20 times (UMPLFLN20XW commercially available from Olympus Corporation) was used as the objective lens 40, an LED (center wavelength of 505 nm) was used as the excitation light source 50, a distributed feedback type (DFB) quantum cascade laser (continuous oscillation type) was used as the infrared light source 20, an objective lens for ZnSe infrared light condensation (model number: #88-447, focal length of 12 mm commercially available from Edmund Optics) was used as the objective lens 70, and a CCD camera (BasleracA 1300-30 um) having 1280×960 pixels (640×480 valid pixels) was used as the imaging device 80. A binning process (2×2) was performed on the imaging device 80 in order to improve an S/N ratio. An observation field of view of the imaging device 80 was 180 μm×135 μm. A gain of the imaging device 80 was 0 dB, and a resolution of the imaging device 80 was 8 bits. A gamma correction of the imaging device 80 was not performed. An exposure time for which the imaging device 80 performed imaging was 400 milliseconds and a frame rate was 2 fps. The excitation filter 51 transmitted light with a wavelength of 489 nm to 505 nm, and the fluorescent filter 81 transmitted light with a wavelength of 524 nm to 546 nm. The dichroic mirror 60 reflected light with a wavelength shorter than a specific wavelength of 515 nm but transmitted light with a wavelength equal to or greater than the specific wavelength.

### FIRST EXAMPLE

In the first example, Hela cells were prepared as the cells 2. The cells 2 were cultured in a Dulbecco's Modified Eagle's medium (DMEM) including 12% fetal bovine serum (FBS) and 4 mM glutamic acid. A dyeing treatment using a fluorescent reagent was performed on the cells 2. As the fluorescent reagent, a calcium fluorescent reagent that quantitatively reacts with Ca³⁺ in the cells 2 and emits the fluorescence L3 was prepared. In the present example, the calcium fluorescent reagent was Calcium Green-1 AM (commercially available from Thermo Fisher Scientific). The calcium fluorescent reagent was excited by the excitation light L2 with a wavelength of 506 nm. A center wavelength of the fluorescence L3 of the calcium fluorescent reagent was 531 nm.

Here, the dyeing treatment method using the calcium fluorescent reagent will be described in detail. First, a solution A (HEPES buffer, total amount of 50 ml) containing 10 mM HEPES, 140 mM NaCl, 4 mM KCl, 2 mM MgCl₂, 2 mM CaCl₂, and 10 mM glucose was prepared. Then, a solution B in which 50 μl of dimethylsulfoxide (DMSO) was added to 50 μg of the calcium fluorescent reagent and dissolved was prepared. Next, a solution C (total amount of 7.75 ml) in which 50 μl of the solution B and 50 μl of a surfactant (Pluronic F127) were added to 7.56 ml of the solution A was prepared. Then, the solution C and the solution A were heated at 37° C. Then, the culture medium (DMEM) was removed from the culture plate 10, and 950 μl of the solution C was added to the culture plate 10. Then, the culture plate 10 to which the solution C was added was incubated at 37° C. for 1 hour. Then, the solution C was removed from the culture plate 10, the cells 2 in the culture plate 10 were washed with the solution A, and 2.0 ml of the solution A was then added to the culture plate 10. According to the above method, the calcium fluorescent reagent was incorporated into the cells 2 and the dyeing treatment was performed.

Next, the excitation light L2 and the mid-infrared light L1 were emitted to the cells 2 on which the dyeing treatment was performed, and the fluorescence L3 was observed. Specifically, first, the excitation light L2 was emitted to the cells 2 by the excitation light source 50. The excitation light L2 passed through the excitation filter 51 and was reflected by the dichroic mirror 60, and then emitted to the cells 2 in the culture plate 10 through the objective lens 40. When the excitation light L2 was emitted to the cells 2, the calcium fluorescent reagent of the cells 2 in the culture plate 10 was excited, and the fluorescence L3 with an intensity corresponding to the Ca²⁺ concentration was emitted from the cells 2. The fluorescence L3 emitted from the cells 2 was collimated by the objective lens 70, then passed through the dichroic mirror 60 and the fluorescent filter 81, and reached the imaging device 80.

Next, the mid-infrared light L1 was emitted to the cells 2 from the infrared light source 20 and emission of the mid-infrared light L1 to the cells 2 was then stopped, and these operations were repeated. That is, an emission period during which the mid-infrared light L1 was emitted to the cells 2 and a non-emission period during which the mid-infrared light L1 was not emitted to the cells 2 were alternately repeated. In the present example, the wavelength of the mid-infrared light L1 was set to 73 um and the emission intensity of the mid-infrared light L1 was set to 30 mW. Then, the emission period was set to 6 seconds, and the non-emission period was set to 8 seconds. In the emission period, the mid-infrared light L1 was incident on the back surface of the silicon wafer 11 of the culture plate 10 through the shutter 30 and the objective lens 40, then passed through the silicon wafer 11, and was continuously emitted to the cells 2 on the silicon wafer 11. Here, when the mid-infrared light L1 was incident on the back surface of the silicon wafer 11, for example, since the energy of the mid-infrared light L1 was reduced due to Fresnel reflection at an interface between air and the silicon wafer 11, the mid-infrared light L1 was thought to be emitted to the cells 2 with an emission intensity of about 13 mW. The emission spot diameter of the mid-infrared light L1 when the mid-infrared light L1 was emitted to the cells 2 was less than 50 μmφ. In the non-emission period, since the mid-infrared light L1 was blocked by the shutter 30, it was not emitted to the cells 2. In this manner, when emission and non-emission of the mid-infrared light L1 to the cells 2 were alternately repeated, changes in the intensity of the fluorescence L3 emitted from the cells 2 were successively observed using the imaging device 80.

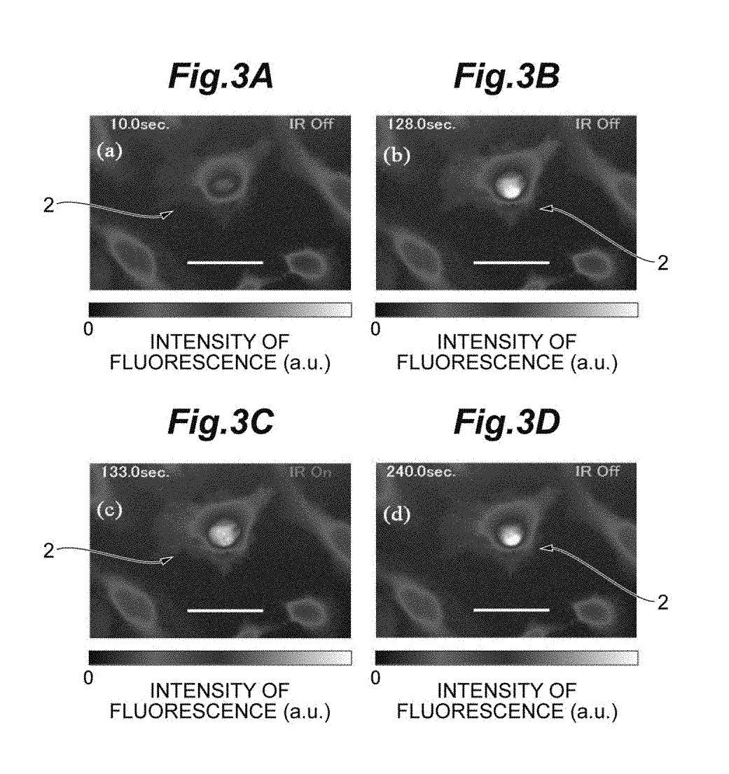

FIG. 3A is an image showing an intensity of the fluorescence L3 before emission of the mid-infrared light L1 to the cells 2 was started (specifically, when 10 seconds had elapsed after the observation was started). FIG. 3B is an image showing an intensity of the fluorescence L3 in the non-emission period (specifically, when 128 seconds had elapsed after the observation was started) during which emission of the mid-infrared light L1 to the cells 2 was stopped after the mid-infrared light L1 was emitted to the cells 2. FIG. 3C is an image showing an intensity of the fluorescence L3 in the emission period (specifically, when 133 seconds had elapsed after the observation was started) during which the mid-infrared light L1 was emitted to the cells 2. FIG. 3D is an image showing an intensity of the fluorescence L3 after repetition of emission and non-emission of the mid-infrared light L1 to the cells 2 was completed (specifically, when 240 seconds had elapsed after the observation was started). In these drawings, the intensity of the fluorescence L3 is indicated by light and dark shading of a color, with lighter shading indicating a higher intensity of the fluorescence L3 and darker shading indicating a lower intensity of the fluorescence L3. The magnitude of the intensity of the fluorescence L3 represents a magnitude of the Ca²⁺ concentration of the cells 2. Therefore, “the intensity of the fluorescence L3” will be appropriately referred to as “Ca²⁺ concentration” in the description.

As shown in FIG. 3A, FIG. 3B, and FIG. 3C, when emission of the mid-infrared light L1 to the cells 2 was started, the Ca²⁺ concentration of the cells 2 clearly increased. Then, as shown in FIG. 3D, it can be understood that, even after repetition of emission and non-emission of the mid-infrared light L1 to the cells 2 was completed, the Ca³⁺ concentration of the cells 2 remained high.

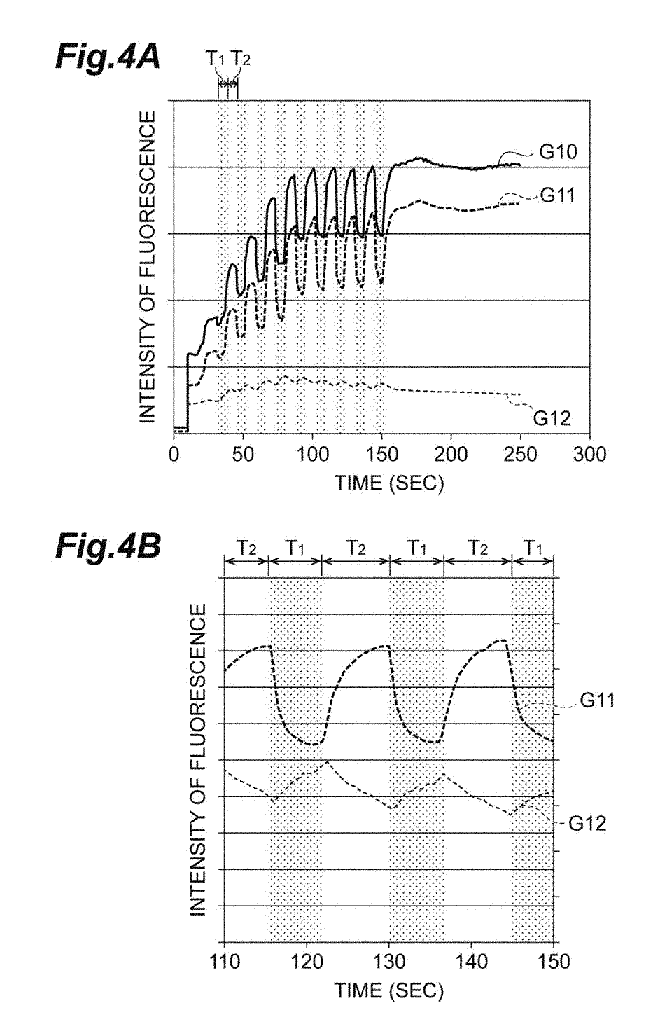

FIG. 4A is a graph showing change in the intensity of the fluorescence L3 over time. FIG. 4B is a graph showing a part (specifically, a time from 110 seconds to 150 seconds) in FIG. 4A enlarged. In FIG. 4A and FIG. 4B, the vertical axis represents the intensity of the fluorescence L3, and the horizontal axis represents a time (sec) after observation was started. In. FIG. 4A and FIG. 4B, G10 shows change in the intensity of the fluorescence L3 of the whole cells 2 over time, G11 shows change in the intensity of the fluorescence L3 in the vicinity of the cell nucleus of the cells 2 over time, and G12 shows change in the intensity of the fluorescence L3 of the cytoplasm of the cells 2 over time. In FIG. 4A and FIG. 4B, an emission period T1 and a non-emission period T2 are alternately repeated.

As shown in FIG. 4A and FIG. 4B, the Ca²⁺ concentration of the whole cells 2 and the Ca²⁺ concentration in the vicinity of the cell nucleus of the cells 2 monotonically decreased in the emission period T1 and monotonically increased in the non-emission period T2. Then, after the repetition of emission and non-emission of the mid-infrared light L1 to the cells 2 was completed, these Ca²⁺ concentrations increased and then remained at a high level. On the other hand, the Ca³⁺ concentration of the cytoplasm of the cells 2 monotonically increased in the emission period T1 and monotonically decreased in the non-emission period T2. In this manner, the Ca²⁺ concentrations increased and decreased in the vicinity of the cell nucleus of the cells 2 and the cytoplasm in an inverse manner. The reason for this is inferred to be that there is an endoplasmic reticulum in which Ca²⁺ ions were stored in the vicinity of the cell nucleus of the cells 2. That is, when the mid-infrared light L1 was emitted to the cells 2, it is thought that Ca²⁺ ions flowed into the cytoplasm from the endoplasmic reticulum. As a result, it is thought that the Ca²⁺ concentration in the vicinity of the cell nucleus of the cells 2 decreased, but the Ca³⁺ concentration in the cytoplasm of the cells 2 increased. In addition, the Ca²⁺ concentration in the vicinity of the cell nucleus of the cells 2 increased overall while repeatedly increasing and decreasing. According to an increase and decrease in the Ca³⁺ concentration in the vicinity of the cell nucleus of the cells 2, the Ca²⁺ concentration of the whole cells 2 increased overall. This is thought to be caused by the fact that Ca²⁺ ions gradually accumulated in the vicinity of the cell nucleus of the cells 2. The reason why Ca²⁺ ions gradually accumulated in the vicinity of the cell nucleus of the cells 2 is thought to be that Ca²⁺ ions flowed in from the outside or there were more Ca²⁺ ions due to calcium being released from the vicinity of the cell nucleus of the cells 2.

### SECOND EXAMPLE

In the second example, the wavelength of the mid-infrared light L1 was set to 6.1 μm and the emission intensity of the mid-infrared light L1 was set to 60 mW. The other conditions were the same as those in the first example.

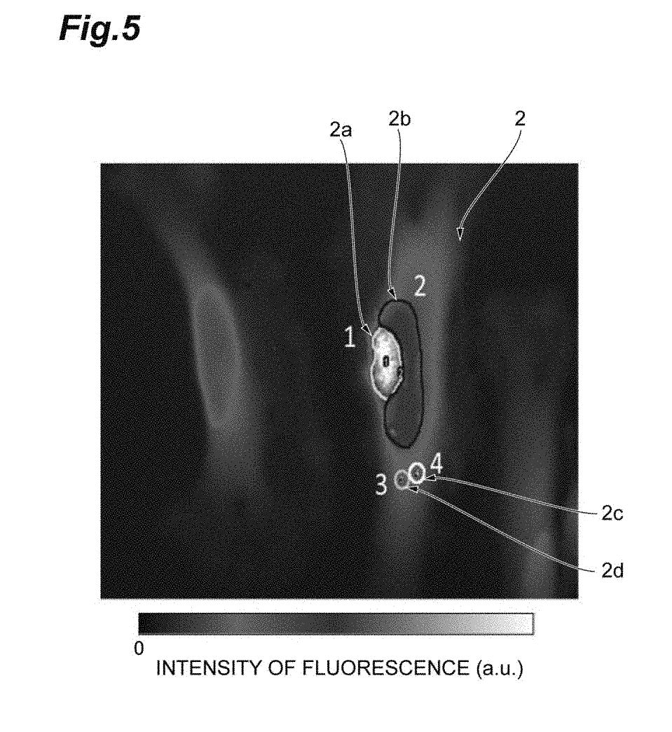

FIG. 5 is an image showing an intensity of the fluorescence L3 in the emission period T1. In FIG. 5, the intensity of the fluorescence L3 is indicated by light and dark shading of a color, with lighter shading indicating a higher intensity of the fluorescence L3 and darker shading indicating a lower intensity of the fluorescence L3. FIG. 5 shows the vicinity 2a of the cell nucleus of the cells 2 (that is, a part of the cell 2) to which the mid-infrared light L1 was continuously emitted, and the vicinity 2b of the cell nucleus of the cells 2, a cytoplasm 2c, and a cytoplasm 2d to which the mid-infrared light L1 was not emitted. As shown in FIG. 5, it can be understood that the Ca³⁺ concentrations in the vicinity 2b of the cell nucleus, the cytoplasm 2c, and the cytoplasm 2d were smaller than the Ca³⁺ concentration in the vicinity 2a of the cell nucleus. Thus, in FIG. 5, parts of the cytoplasm 2c with a high Ca²⁺ concentration were scattered in spots. That is, in the cytoplasm 2c, Ca²⁺ ions accumulated in spots.

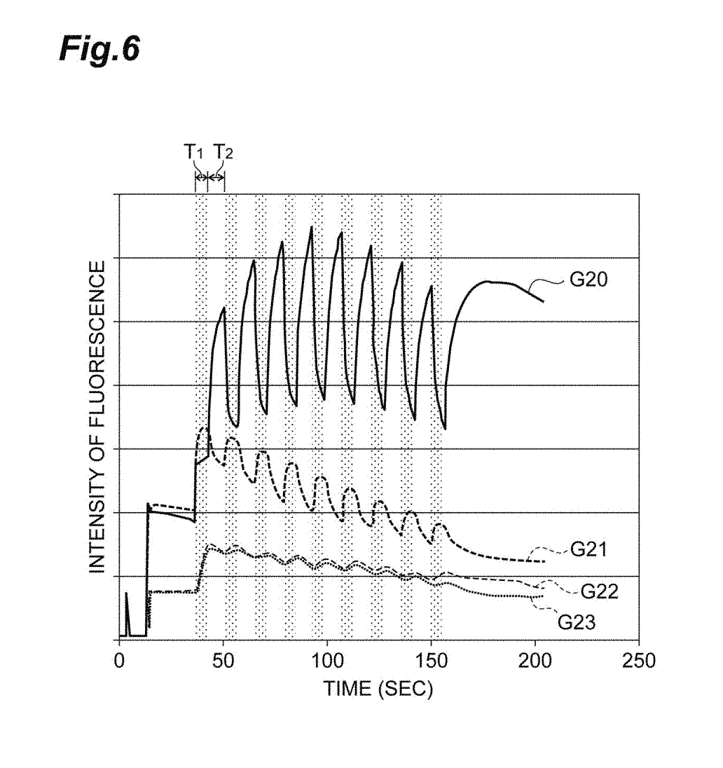

FIG. 6 is a graph showing change in the intensity of the fluorescence L3 over time. In FIG. 6, the vertical axis represents the intensity of the fluorescence L3 and the horizontal axis represents a time (sec) after observation was started. G20 shows change in the intensity of the fluorescence L3 in the vicinity 2a of the cell nucleus over time, G21 shows change in the intensity of the fluorescence L3 in the vicinity 2b of the cell nucleus over time, G22 shows change in the intensity of the fluorescence L3 of the cytoplasm 2c over time, and G23 shows change in the intensity of the fluorescence L3 of the cytoplasm 2d over time. In FIG. 6, the emission period T1 and the non-emission period T2 are alternately repeated. As shown in FIG. 6, the Ca²⁺ concentration in the vicinity 2a of the cell nucleus monotonically decreased in the emission period T1 and monotonically increased in the non-emission period T2. Then, after repetition of emission and non-emission of the mid-infrared light L1 to the cells 2 was completed, the Ca³⁺ concentration in the vicinity 2a of the cell nucleus increased and was then remained at a high level. On the other hand, the Ca²⁺ concentrations in the vicinity 2b of the cell nucleus, the cytoplasm 2c, and the cytoplasm 2d monotonically increased in the emission period T1 and monotonically decreased in the non-emission period T2. Here, during the intermediate period from when repetition of emission and non-emission of the mid-infrared light L1 to the cells 2 was started, the Ca²⁺ concentration of the cytoplasm 2c and the Ca²⁺ concentration of the cytoplasm 2d were changed similarly. However, after this period had passed, the Ca³⁺ concentration of the cytoplasm 2c was larger than the Ca²⁺ concentration of the cytoplasm 2d, and a difference between the Ca³⁺ concentration of the cytoplasm 2c and the Ca²⁺ concentration of the cytoplasm 2d gradually increased.

### THIRD EXAMPLE

In the third example, CHO cells were prepared as the cells 2. In addition, the wavelength of the mid-infrared light L1 was set to 6.1 μm. The other conditions were the same as those in the first example.

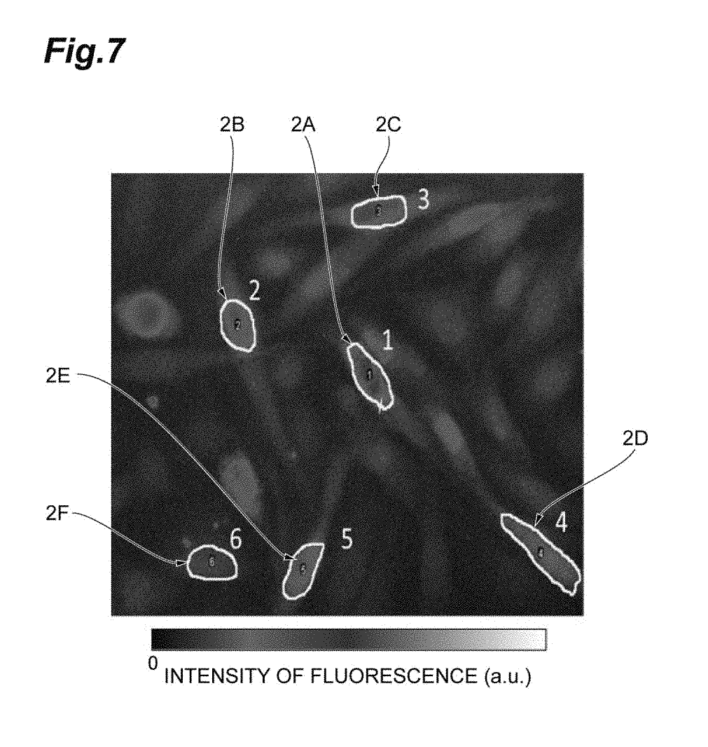

FIG. 7 is an image showing an intensity of the fluorescence L3 in the emission period T1. In FIG. 7, the intensity of the fluorescence L3 is indicated by light and dark shading of a color, with lighter shading indicating a higher intensity of the fluorescence L3 and darker shading indicating a lower intensity of the fluorescence L3. FIG. 7 shows cells 2A and other cells 2B to 2F disposed around the cells 2A. The mid-infrared light L1 was continuously emitted to the cells 2A but it was not emitted to the cells 2B to 2F. As shown in FIG. 7, it can be understood that not only the Ca²⁺ concentration of the cells 2 to which the mid-infrared light L1 was emitted but also the Ca³⁺ concentrations of the cells 2B to 2F to which the mid-infrared light L1 was not emitted increased. In addition, in FIG. 7, it was observed that the Ca²⁺ concentration in the vicinity of the cell nucleus of the cells 2A was higher than the Ca²⁺ concentration in other parts of the cells 2A.

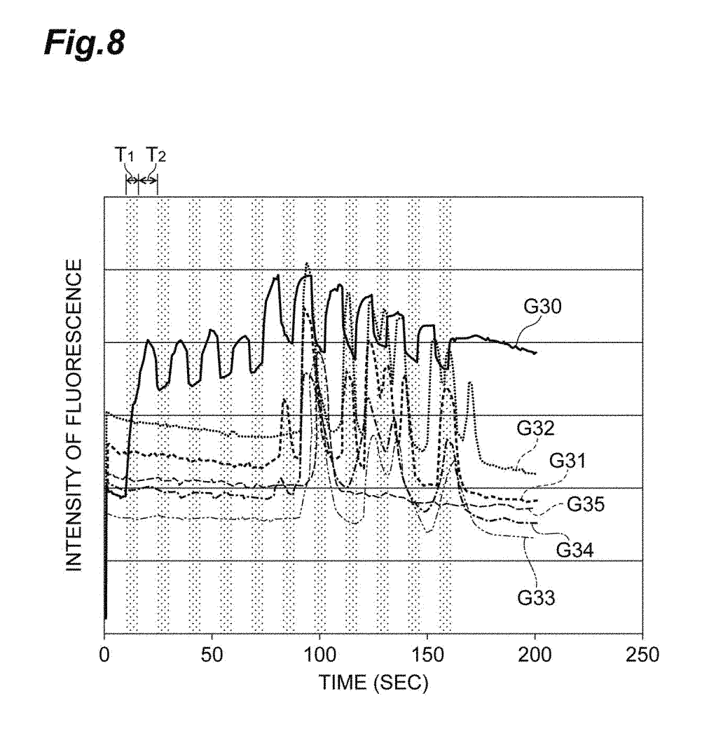

FIG. 8 is a graph showing change in the intensity of the fluorescence L3 over time. In FIG. 8, the vertical axis represents the intensity of the fluorescence L3 and the horizontal axis represents a time (sec) after observation was started. G30 to G34 show change in the intensity of the fluorescence L3 of the cells 2A to 2F over time, respectively. In FIG. 8, the emission period T1 and the non-emission period T2 are alternately repeated. As shown in FIG. 8, the Ca²⁺ concentration of the cells 2A monotonically decreased in the emission period T1 and monotonically increased in the non-emission period T2. Then, after the repetition of emission and non-emission of the mid-infrared light L1 to the cells 2A was completed, the Ca²⁺ concentration of the cells 2A increased and then remained at a high level. It was observed that the Ca³⁺ concentrations of the cells 2B to 2F started to increase and decrease from the non-emission period T2 in which repetition of emission and non-emission of the mid-infrared light L1 to the cells 2A was being performed. This is thought to be caused by the fact that Ca²⁺ ions in the cells 2A are transmitted to the surrounding cells 2B to 2F. The Ca³⁺ concentrations of the cells 2B to 2F increased and decreased at timings that were different from timings at which the mid-infrared light L1 was emitted or not emitted to the cells 2A. Timings at which the Ca³⁺ concentrations of the cells 2B to 2F increased and decreased were different from each other.

### FOURTH EXAMPLE

In the fourth example, as the fluorescent reagent, a sodium fluorescent reagent that quantitatively reacts with Na⁺ in the cells 2 and emits the fluorescence L3 was prepared. The sodium fluorescent reagent was CoroNa AM (commercially available from Thermo Fisher Scientific). The sodium fluorescent reagent was excited by the excitation light L2 with a wavelength of 492 nm. The center wavelength of the fluorescence L3 of the sodium fluorescent reagent was 516 nm. A dyeing treatment using the sodium fluorescent reagent was performed on the cells 2.

Here, the dyeing treatment method using the sodium fluorescent reagent will be described in detail. First, a solution. Al (HEPES buffer, total amount of 50 ml) containing 10 mM HEPES, 140 mM NaCl, 4 mM KCl, 2 mM MgCl₂, 2 mM CaCl₂, and 10 mM glucose was prepared. In addition, a solution B1 in which 50 μl of dimethylsulfoxide (DMSO) was added to 50 μg of the sodium fluorescent reagent and dissolved was prepared. Next, a solution C1 (total amount of 7.75 ml) in which 50 μl of the solution B1 and 50 μl of a surfactant (Pluronic F127) were added to 7.56 ml of the solution A1 was prepared. Then, the solution C1 and the solution A1 were heated a 37° C. Then, the culture medium (DMEM) was removed from the culture plate 10 and 950 μl of the solution C1 was added to the culture plate 10. The culture plate 10 to which the solution C1 was added was incubated at 37° C. for 45 minutes. Then, the solution C1 was removed from the culture plate 10, the cells 2 in the culture plate 10 were washed with the solution A1, and 2.0 ml of the solution A1 was then added to the culture plate 10. According to the above method, the sodium fluorescent reagent was incorporated into the cells 2, and the dyeing treatment was performed. In addition, the emission intensity of the mid-infrared light L1 was set to 60 mW. The other conditions were the same as those in the first example.

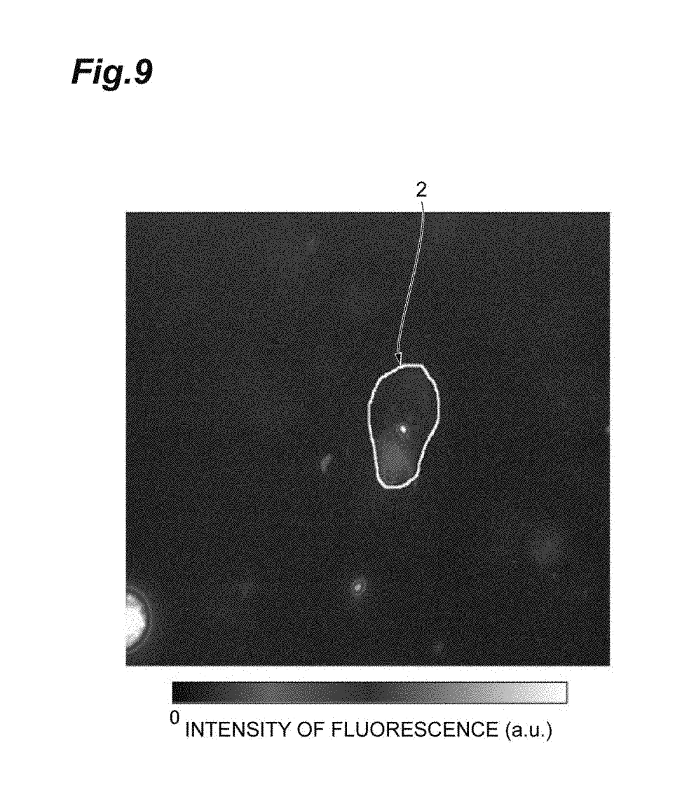

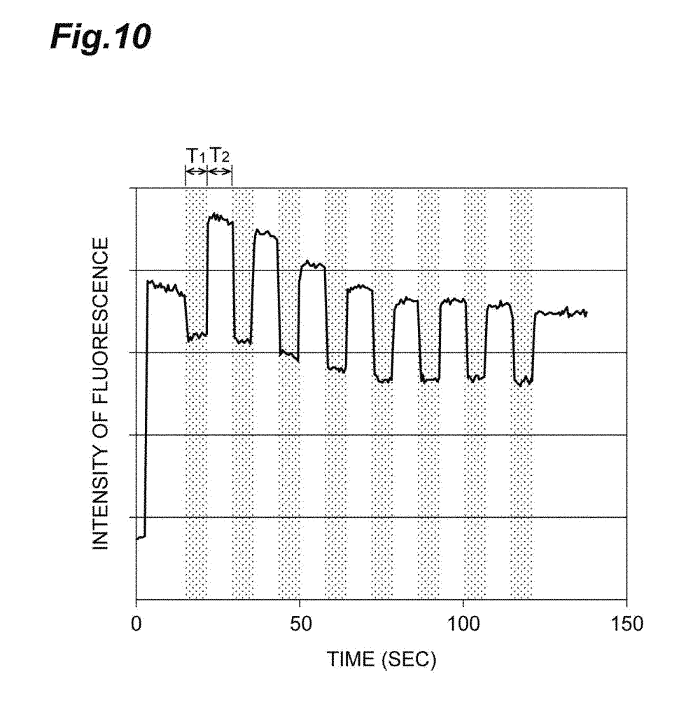

FIG. 9 is an image showing an intensity of the fluorescence L3 in the emission period T1. In FIG. 9, the intensity of the fluorescence L3 is indicated by light and dark shading of a color, with lighter shading indicating a higher intensity of the fluorescence L3 and darker shading indicating a lower intensity of the fluorescence L3. The magnitude of the intensity of the fluorescence L3 represents a magnitude of the Na⁺ concentration of the cells 2. Therefore, “the intensity of the fluorescence L3” will be appropriately referred to as “Na⁺ concentration” in the description. FIG. 9 shows the cells 2 to which the mid-infrared light L1 was continuously emitted. As shown in FIG. 9, it can be understood that, when the mid-infrared light L1 was continuously emitted to the cells 2, the Na⁺ concentration increased. FIG. 10 is a graph showing change in the intensity of the fluorescence L3 over time. In FIG. 10, the vertical axis represents the intensity of the fluorescence L3 and the horizontal axis represents a time (sec) after observation was started. In FIG. 10, the emission period T1 and the non-emission period T2 are alternately repeated. As shown in FIG. 10, the Na⁺ concentration of the cells 2 monotonically decreased in the emission period T1 and monotonically increased in the non-emission period T2. Then, after the repetition of emission and non-emission of the mid-infrared light L1 to the cells 2 was completed, the Na⁺ concentration of the cells 2 increased and was then remained at a high level. Therefore, when the mid-infrared light L1 was continuously emitted to the cells 2, the Na⁺ concentration of the cells 2 was changed in the same manner as the Ca²⁺ concentration of the cells 2.

Next, effects obtained by the cell stimulation device 1 and the cell stimulation method according to the above embodiment, and the first example to the fourth example will be described with reference to the related art.

Biological cells include organic molecules (biomolecules) such as nucleic acids, proteins, lipids, and sugars. A balance of physiological functions of such biomolecules is generally maintained according to interactions such as functional groups of biomolecules and bonding between the biomolecules. However, for example, when the balance of physiological functions is disturbed by an external factor, various diseases may be caused in a living body. Accordingly, it is desirable that the balance of physiological functions be maintained in the living body. Here, the physiological functions are functions in a living body, for example, contraction of muscle cells, signal transmission of cells, or functional regulation of proteins. Such physiological functions are controlled by, for example, ions of biomolecules. For example, Ca²⁺ ions play an important role as a component that transmits signals of cells. For example, in the contraction of muscle cells, calcium may function as a component controlling physiological functions and biomolecules downstream of a signal of Ca²⁺ may exhibit various physiological functions. The physiological functions may be controlled by not only Ca²⁺ of biomolecules but also other ions of biomolecules. Therefore, it is thought that, even if the balance of physiological functions is disturbed, when an ion concentration of biomolecules is intentionally changed, the balance of physiological functions can be maintained again. That is, it is thought that, when an ion concentration of biomolecules in cells can be intentionally changed, it is possible to maintain a state in which the balance of physiological functions is maintained, and there is a possibility of the occurrence of various diseases in a living body being prevented.

Therefore, as a method of intentionally changing an ion concentration of biomolecules, a method in which infrared light is emitted to biomolecules in cells and thus an ion concentration of biomolecules is changed may be conceived. When infrared light is emitted to biomolecules in cells, the biomolecules absorb infrared light photon energy. A magnitude of infrared light photon energy absorbed by the biomolecules corresponds to a magnitude of the energy necessary to change a vibration state of the biomolecules. Therefore, when infrared light is emitted to biomolecules, it is possible to change a vibration state of the biomolecules. Such a change in the vibration state of the biomolecules is thought to cause a change in an ion concentration of the biomolecules. For example, a method in which the Ca²⁺ concentration in cells is changed using near infrared light has been conceived (for example, refer to Non-Patent Literature 1 and Non-Patent Literature 2). However, since not much light with a wavelength in a near infrared range is absorbed by biomolecules, it is difficult to efficiently change an ion concentration of living cells using near infrared light.

On the other hand, the inventors focused on the fact that a wavelength range of mid-infrared light within infrared light L1 corresponds to a fingerprint range of biomolecules (that is, a wavelength range in which intrinsic absorption peaks of biomolecules appear), and is a wavelength range in which absorption into biomolecules in the cells 2 is greatest, and found that, when the mid-infrared light L1 is directly emitted to the cells 2, it is possible to efficiently change an ion concentration of the living cells 2. Specifically, since many intrinsic absorption peaks of biomolecules in the cells 2 appear in the wavelength range of the mid-infrared light L1, when the mid-infrared light L1 having a wavelength corresponding to an absorption band of a certain specific biomolecule is emitted to the cells 2, it is possible to change an ion concentration of an arbitrary biomolecule in the cells 2. Thus, in contrast to near infrared light, since absorption into biomolecules in the cells 2 is greatest in the mid-infrared light L1, even if an emission intensity of the mid-infrared light L1 is reduced to become lower than an emission intensity (that is, an emission intensity necessary for changing an ion concentration of the cells 2) of near infrared light, it is possible to change an ion concentration of the cells 2. In addition, since an emission intensity of the mid-infrared light L1 can be reduced in this manner, even if the mid-infrared light L1 is continuously emitted to the cells 2, it is possible to change an ion concentration of the cells 2 while avoiding damage to the cells 2 or cell death. Therefore, it is possible to sustainably change an ion concentration of the cells 2.

As in the above embodiment and the first example to the fourth example, the wavelength of the mid-infrared light L1 may be 4 μm or more and 10 μm or less. Many intrinsic absorption peaks of biomolecules in the cells 2 appear particularly in this wavelength range. Accordingly, the above-described effects can be suitably obtained.

As in the second example, the mid-infrared light L1 may be emitted to the vicinity 2a of the cell nucleus which is a part of the cell 2. In this manner, when the mid-infrared light L1 is locally emitted to the vicinity 2a of the cell nucleus, a change in the Ca²⁺ concentration that is different from changes in the Ca²⁺ concentrations of the vicinity 2b of the cell nucleus, the cytoplasm 2c, and the cytoplasm 2d can be caused in the vicinity 2a of the cell nucleus. That is, it is possible to locally change the Ca²⁺ concentration of the cells 2.

The cell stimulation method and the cell stimulation device according to the present disclosure are not limited to those of the embodiment, and the first example to the fourth example described above, and various other modifications can be made. For example, while the mid-infrared light L1 has been emitted to the cells 2 in the culture plate 10 in the embodiment, and the first example to the fourth example described above, the mid-infrared light L1 may be emitted to cells in a living body.

What is claimed is:

1. A cell stimulation method comprising:
continuously emitting mid-infrared light to a living cell and thus changing an ion concentration of the cell or changing ion concentrations of the cell and other cells disposed around the cell.

2. The cell stimulation method according to claim 1,
wherein the mid-infrared light is emitted to a part of the cell.

3. The cell stimulation method according to claim 1,
wherein a wavelength of the mid-infrared light is 4 μm or more and 10 μm or less.

4. The cell stimulation method according to claim 2,
wherein a wavelength of the mid-infrared light is 4 μm or more and 10 μm or less.

5. A cell stimulation device comprising:
a light emission unit configured to output mid-infrared light,
wherein, when the mid-infrared light is continuously emitted to a living cell, an ion concentration of the cell is changed or ion concentrations of the cell and other cells disposed around the cell are changed.
